(19) United States
(12) Patent Application Publication
(10) Pub. No.: US 2024/0108270 A1
(43) Pub. Date: Apr. 4, 2024

(54) METHOD AND SYSTEM FOR ESTIMATING THE EXTENT OF ISCHEMIA IN AN INDIVIDUAL

(71) Applicant: John Heibel, Baldwin, MI (US)

(72) Inventor: John Heibel, Baldwin, MI (US)

(21) Appl. No.: 17/958,716

(22) Filed: Oct. 3, 2022

(51) Int. Cl. A61B 5/347; A61B 5/00; A61B 5/0245; G16H 50/30

(52) U.S. Cl. CPC A61B 5/347; A61B 5/0245; A61B 5/7246; A61B 5/7278; G16H 50/30

(57) ABSTRACT

A method and system for estimating the extent of ischemia in a test individual using an electrocardiogram (EKG) apparatus. An EKG is obtained from the test individual. A test EKG dataset is obtained comprising EKG measurements from the EKG. The test EKG dataset is converted to a frequency domain. An area under the curve is measured of the test EKG dataset in the frequency domain with respect to a reference EKG dataset comprising EKG measurements in the frequency domain. The area under the curve measurement is normalized to calculate a percentage score value to estimate an extent of ischemia in the test individual.

### FIELD AND BACKGROUND OF THE INVENTION

The present disclosure generally relates to a method and system for estimating early ischemia in an individual based on data collected from the individual's heartbeat.

A medical doctor, such as a cardiologist, can detect a fully developed infarction or severe ischemia from the shape of an electrocardiogram reading (known as an ECG or EKG, but hereinafter referred to as an EKG). An infarction is the injury or death of tissue or an organ (such as the heart or lungs) from a loss or inadequate supply of blood due to obstructed or narrowed blood vessels. Ischemia is a condition where tissue or an organ experiences injury from a diminished blood flow due to a partial blockage or narrowing of blood vessels. Ischemia, and a partial blockage, can progress or worsen to an infarction and the obstruction or narrowing of blood vessels. Thus, the detection of ischemia before it reaches the level of an infarction is important, because a myocardial infarction (a heart attack), is one of the leading causes of death.

However, an EKG, while useful for monitoring a person's heart and for detecting health issues, is unable to reliably detect ischemia in the early stages (where it is assumed that early treatment could prevent further health complications). In general, it is much more difficult to detect ischemia in the early stages as opposed to a more advanced stage of ischemia or an infarction. Other methods or techniques for detecting ischemia (besides evaluating an EKG) include, for example, blood tests, chest X-rays, a cardiac stress test, a coronary CT angiogram, and a magnetic resonance angiogram. Thus, there is a need for improved methods for detecting ischemia in the early stages.

### SUMMARY OF THE INVENTION

What is provided is an improved method and system for quickly, efficiently, reliably, and accurately estimating the extent of ischemia in an individual by using data collected from the individual's heartbeat. As a result, data and information is obtained, computed, and analyzed from an electrocardiogram (EKG) of an individual, which is used as the source for ischemia calculations and predictions for the individual. The data from the EKG of the individual is compared to a reference EKG which is from another individual with no ischemia.

A method and system for estimating the extent of ischemia in a test individual, according to an aspect of the invention, includes using an electrocardiogram (EKG) to obtain a dataset of EKG measurements captured from the test individual, and using a computer-based system that is programmed with computer code to convert the dataset of EKG measurements captured from the test individual to a frequency domain. The dataset of EKG measurement in the frequency domain of the test individual is compared to a reference dataset of EKG measurements in the frequency domain of a reference individual to estimate the extent of ischemia in the test individual.

In an embodiment, a computer system is used to estimate the extent of ischemia in the individual. Software programs, such as MATLAB®, operating on the computer system may be used to conduct various computing, calculating, and analyzing steps in the process of estimating the extent of ischemia in the individual.

In an embodiment, a method and system for estimating the extent of ischemia in a test individual includes using an electrocardiogram (EKG) apparatus to obtain an EKG from the test individual. A test EKG dataset is obtained comprising selected EKG measurements from the EKG. The test EKG dataset is converted to a frequency domain. An area under the curve is measured of the test EKG dataset in the frequency domain with respect to a reference EKG dataset comprising selected EKG measurements in the frequency domain. The area under the curve measurement is normalized to calculate a percentage score value to estimate an extent of ischemia in the test individual.

In an embodiment, a system for estimating the extent of ischemia in a test individual includes an electrocardiogram (EKG) apparatus and a computer-based system. The EKG apparatus is configured to obtain an EKG of the test individual. The computer-based computer is programmed with computer code configured to obtain a test EKG dataset comprising selected EKG measurements from the EKG. The computer-based system converts the test EKG dataset to a frequency domain and measures an area under the curve of the test EKG dataset in the frequency domain with respect to a reference EKG dataset comprising EKG measurements in the frequency domain. The computer-based system is also configured to normalize the area under the curve measurement to calculate a percentage score value used to estimate an extent of ischemia in the test individual.

In an aspect of an embodiment, the reference individual is an individual that is substantially free of ischemia.

In another aspect of an embodiment, the computer-based system is configured to normalize the area under the curve measurement with respect to a reference EKG dataset of an individual that has a diagnosis of an infarction to define the extent of ischemia in the test individual.

In a further aspect of an embodiment, a diagnosis of infarction is present when the calculated percentage score value is 100%. The percentage score value also indicates an estimation of ischemia present in the test individual when the calculated percentage score value is at least 10%.

In another aspect of an embodiment, the computer-based system is configured to select a first heartbeat waveform from the EKG for the test EKG dataset, and the first heartbeat waveform is a single heartbeat. The computer-based system is further configured to select a second heartbeat waveform from the EKG for the test EKG dataset. The second heartbeat waveform is identical to the first heartbeat waveform and is concatenated to the first heartbeat waveform.

In yet another aspect of an embodiment, the computer-based system is configured to calculate a difference between amplitudes of a first 50 harmonics of the test EKG dataset in the frequency domain and a first 50 harmonics of the reference EKG dataset in the frequency domain and to estimate an extent of hyperkalemia instead of ischemia when the difference is a reduction in amplitudes of the first 50 harmonics in the test EKG dataset in the frequency domain of at least 60%.

These and other objects, advantages, purposes, and features of this invention will become apparent upon review of the following specification in conjunction with the drawings.

### BRIEF DESCRIPTION OF THE DRAWINGS

The present invention will now be described with reference to the accompanying figures, wherein the numbered elements in the following written description correspond to like-numbered elements in the figures.

FIG. 1 illustrates a block diagram of an exemplary computer system, in accordance with various embodiment of the present disclosure;

FIG. 2A illustrates a plot of a conventional electrocardiograph (EKG) waveform;

FIG. 2B illustrates a plot of the EKG waveform of FIG. 2A converted into a frequency domain;

FIG. 3 illustrates an exemplary method for estimating the extent of ischemia in an individual using the computer system illustrated in FIG. 1;

FIG. 4 illustrates an exemplary plot showing a difference in amplitudes between a first test individual and a reference individual in accordance with the present invention;

FIG. 5 illustrates another exemplary plot showing a difference in amplitudes between the first individual and the reference individual of FIG. 4;

FIG. 6 illustrates an exemplary plot showing a difference in amplitudes between a second test individual with hyperkalemia and the reference individual in accordance with the present embodiment; and

FIG. 7 illustrates another exemplary plot showing the amplitude differences between the second test individual with hyperkalemia and the reference individual of FIG. 6.

### DETAILED DESCRIPTION OF THE PREFERRED EMBODIMENT

The present invention will now be described with reference to the accompanying figures, wherein the numbered elements in the following written description correspond to like-numbered elements in the figures.

Exemplary embodiments of the present invention provide for an estimation of the extent of ischemia in an individual. Because an early warning is not possible after the extent of ischemia in an individual has advanced enough to become an infarction, it is beneficial to detect or estimate the extent of ischemia when it is considered an “early” diagnosis of ischemia. As described herein, an extent of ischemia in a test individual can be determined by comparing the calculated results of an EKG dataset from the test individual to an EKG dataset from a reference individual. The calculated results are normalized with respect to an infarction, such that a normalized maximum value (e.g., 1) correlates to a diagnosis of an infarction, while a normalized value less than maximum is used to define the extent of ischemia in the test individual. That is, the resulting value will fall between 0 and 1. In an aspect of an embodiment, the calculated results of the individual are normalized with respect to a set of calculated results of a reference individual with a diagnosis of an infarction. That is, there is a threshold calculated result that equates to an infarction.

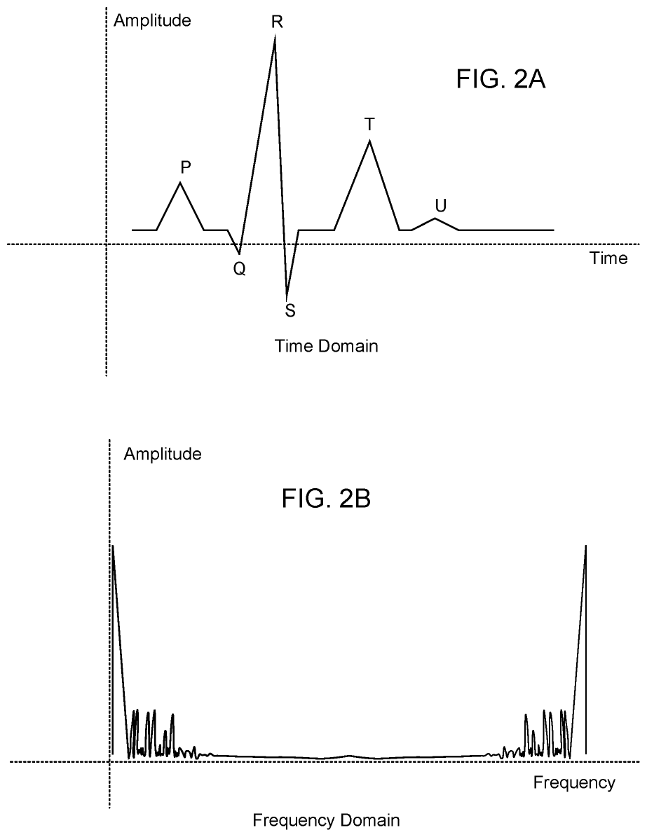

As illustrated in FIG. 2A, an exemplary EKG plot includes component waves that correspond to positive or negative deflections from baseline that indicate specific electrical events. The EKG is an amplitude/time plot. In an embodiment, the waves on an EKG include the P wave, Q wave, R wave, S wave, T wave, and U wave (see FIG. 2A). Such conventional EKG plots are used by physicians (e.g., a cardiologist) to diagnose ischemia or infarctions. By evaluating the shapes and layouts of the waveforms in the EKG plot, the physician can detect and diagnose ischemia and infarction. However, in the early stages of ischemia, the changes in the waveform of an EKG plot are very minute and difficult to detect. When a physician evaluates an EKG plot for heart problems or related cardiovascular issues, the physician is looking for changes to the shape or amplitude of the individual waves in the heartbeat waveform. There are at least seven different waveform changes that can be used to identify where an ischemia is located. For example, when the amplitude value of the line between the S and T waves drops, an ischemia can be diagnosed. When other wave components disappear from the EKG plot, an ischemia can be identified as taking place. However, in general, by the time that the physician can see the changes in the waveform shape in the EKG plot, the ischemia is often well developed and possibly even advanced enough to cause an infarction. That is, an ischemia begins with little to no changes in the waveform of the EKG plot. The first changes for mild ischemia can not be easily detected in the EKG plot. Because of the difficulties in detecting such minute changes in the EKG plot, it is difficult to diagnose ischemia before the disease becomes quite advanced or even progressed to an infarction (tissue death due to complete blood vessel blockage). It is estimated that even a skilled cardiologist can have difficulty in detecting ischemia before the ischemia would result in a percentage score of at least 50% (halfway between a percentage score of 0% (no ischemia) and a percentage score of 100% (an infarction).

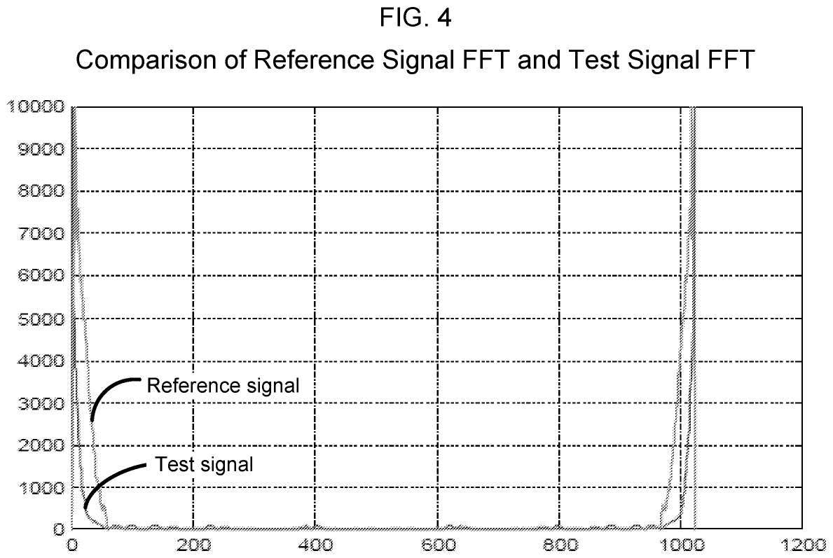

It is also seen that conventional waveshape identification algorithms have not been successful in identifying meaningful changes in an EKG plot. Because of the tolerances that have to be defined in waveshape identification algorithms, such conventional algorithms do not appear to improve upon the diagnosing success of a skilled cardiologist. Note that EKG plots are time domain plots of amplitude changes over time (FIG. 2A). However, because a waveform pattern can be evaluated in either the time domain or frequency domain, in an embodiment, the conventional EKG plot (see FIG. 2A) is converted into the frequency domain (see FIG. 2B). As illustrated in FIGS. 2B, 4, and 5, the frequency domain plot includes a series of harmonics of a fundamental frequency. As illustrated in FIG. 4, the amplitude of the harmonics in a normal EKG (converted to the frequency domain) will normally die off after the first few harmonics. This is seen in the reference EKG plot. Thus, a normal EKG will have expected amplitudes in the first few harmonics, but then little to no amplitude after the next 20-60 frequency harmonics.

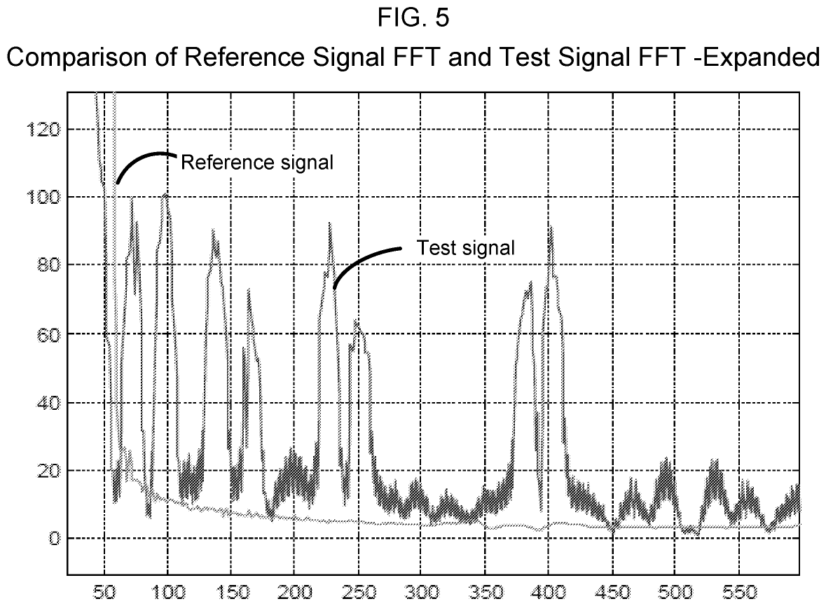

As illustrated in FIGS. 4 and 5, as soon as a person begins developing ischemia, the later harmonics will begin to develop measurable amplitudes (with respect to a reference EKG). As ischemia begins to develop and progress, the harmonics will begin with small amplitudes that grow in amplitude until they are noticeable. A measure of the amplitude of the harmonics can be merely an evaluation of the amplitudes of the harmonics of the test EKG plot with respect to the amplitudes of the harmonics of the reference EKG plot. Alternatively, the measure of the amplitudes of the harmonics can be performed as an “area under the curve” measurement that calculates a two-dimensional area between the test EKG plot and the reference EKG plot. An exemplary “area under the curve” measurement is performed by the processor 114 with the use of a MATLAB® program. Once the area under the curve measurement has been performed, the resulting measurement is normalized with respect to a maximum expected or maximum relevant area under the curve value (e.g., an area under the curve measurement found in individual with an infarction) to estimate the extent of ischemia in the test individual. For example, a normalized value of 1 or 100%=an infarction, while a normalized value of 0.10 or 10%=mild ischemia. Thus, the percentage score is used to estimate the extent of ischemia in the test individual. The higher the area under the curve measurement (and the higher the test EKG dataset diverges from the reference EKG dataset (see FIGS. 4 and 5)), the higher the estimated extent of ischemia or infarction. Thus, a percentage score of 100% is an indication that there is an infarction in the test individual, while a percentage score of at least 10% is an early indication of ischemia. Percentage scores between 10% and 100% are estimations or indications of varying degrees of severity in ischemia and/or infarction. Note that it is an aspect of the present embodiment that a measurable percentage score of 10% provides an estimate of mild ischemia. Such a diagnosis, while visible in the EKG plot's frequency domain is not yet apparent in the EKG plot's time domain.

Accordingly, in an embodiment, a percentage score of 5-10% provides an estimate or indication of early or mild ischemia. While a diagnosis of ischemia (and its location) can not be determined by a conventional time domain EKG plot, an exemplary percentage score of 5-10% could be used by a physician to diagnosis ischemia in the test individual before any other method of diagnosis. As discussed herein, using conventional means, a diagnosis of ischemia by a cardiologist when compared to the exemplary embodiment would result in a percentage score of 30% to 50%. Thus, while undiagnosable using conventional means, an early diagnosis of ischemia according to the exemplary embodiments could provide the physician with an opportunity to start treatment early. While a percentage score that indicates that ischemia is present in the test individual will not identify where the ischemia is located in the test individual, the physician could eventually use a conventional EKG evaluation to look for where the ischemia is (after the ischemia has progressed).

Once a physician can identify that an ischemia exists (using the exemplary embodiments), they can begin looking for where the ischemia is, as well as beginning an early treatment. If the physician knows to look for wave changes in an EKG plot with respect to a reference EKG plot, it might be easier to identify the where of the ischemia via the time domain EKG waveform and its changes due to the ischemia. Furthermore, once ischemia is identified in the frequency domain plot of the EKG, the physician can find and treat the ischemia before it becomes an infarction (which could result in a heart attack if the infarction is in the heart). As noted, the ischemia identified in the frequency domain EKG plot can be identified in a normal time domain EKG plot once it's around a percentage score of 30-40%. That is, a physician could treat it generally until it progresses to the point that the physician identifies where the ischemia is and directly treats its location. The test individual with an indication of ischemia could be given a generalized treatment (e.g., blood thinners or other similar treatments) early even if the physician does not know where in the body the ischemia is. These would be judgement calls by the physician. The physician could receive a percentage score of ischemia as early as 10% (or less) but will not know where the ischemia is until the percentage score reaches as high as 30-40%.

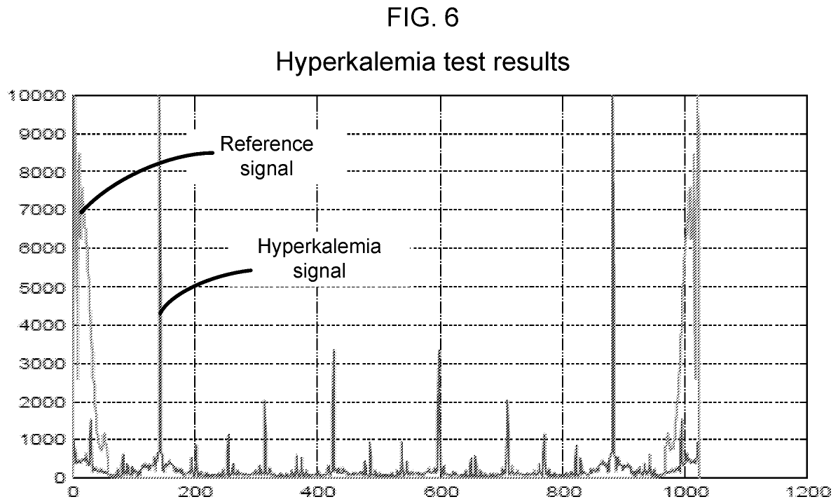

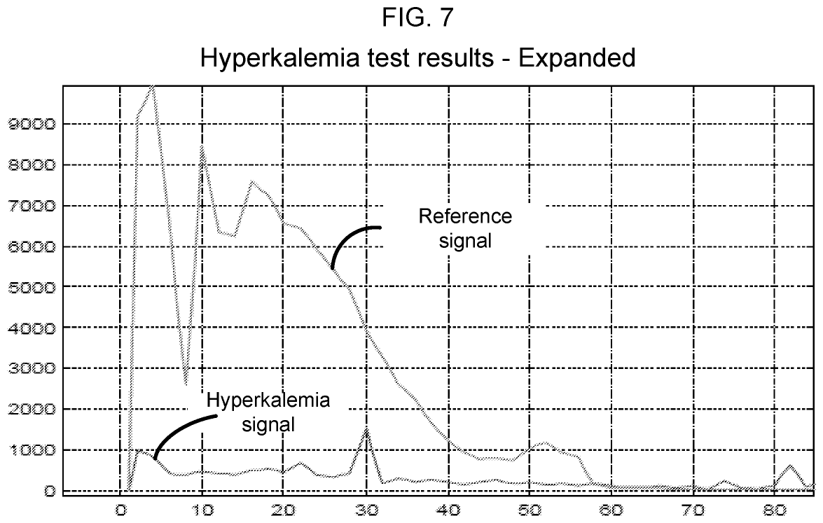

As also discussed herein, hyperkalemia in the test individual (high levels of potassium in the blood) can also result in a high percentage score when evaluating the individual's test EKG plot against a reference EKG plot (both in the frequency domain). However, hyperkalemia results in lower amplitudes in the first 50 harmonics than would be seen in the reference EKG plot (see FIG. 7). FIG. 6 illustrates that hyperkalemia produces the same kind of amplitudes or frequency “bumps” that are produced by ischemia and/or an infarction. Without a further evaluation, the EKG plot could only be used to indicate the presence of ischemia, infarction, or hyperkalemia. As illustrated in FIG. 7, the first 50 harmonics in the EKG plot of the individual with hyperkalemia are shrunk by approximately 90% as compared to the reference EKG plot. Note that the reference EKG plot is from an individual without ischemia and without hyperkalemia. That is, when a percentage score calculation (the area under the curve measurement) indicates ischemia or infarction, if the first 50 harmonics are reduced in amplitude (e.g., by 90%) with respect to the reference EKG plot, the indication is for hyperkalemia instead of ischemia, but if the first 50 harmonics have expected amplitudes, the indication is for ischemia or an infarction (instead of hyperkalemia).

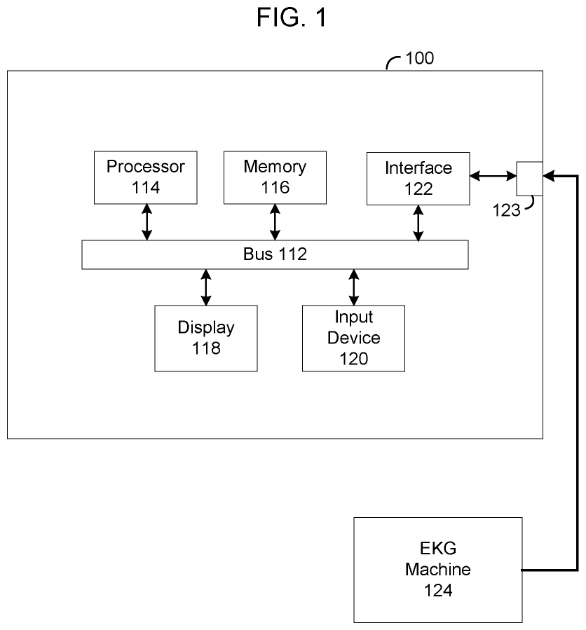

FIG. 1 is a block diagram illustrating an exemplary computer system 100 upon which embodiments of the present disclosure may be implemented, including estimating early ischemia in an individual based on data collected from that individual's heartbeat. The computer system 100 includes a bus 112 or other communication mechanism for communicating information, and a processor 114 coupled with the bus 112 for processing information. The computer system 100 includes a memory 116, which may be a random-access memory (RAM), read only memory (ROM), and/or other dynamic storage device, coupled to the bus 112 for storing instructions to be executed by the processor 114. As discussed herein, the memory 116 may also be used for storing EKG datasets (selected heartbeat recordings).

The computer system 100 may be coupled to a display 118 for displaying information to a computer user. An input device 120 is coupled to the bus 112 for communicating information and command selections to the processor 114. The input device 120 may, for example, be a mouse, a trackball, or a cursor for communicating direction information and command selections to the processor 114. An interface 122 is coupled to the 112 for communicating data to the processor 114. For example, a device 124 capable of capturing an electrocardiogram (e.g., an EKG machine) is communicatively coupled to the interface 122 (via a port 123) to communicate the electrocardiogram to the processor 114. EKG datasets are then captured by the processor 114 and stored in memory 116. Alternatively, EKG datasets could be provided by the EKG machine 124 itself, or from other sources coupled to the interface (e.g., an external memory device) 122. These datasets would also be stored in the memory 116 for processing by the processor 114.

Consistent with certain implementations of the present disclosure, results are provided by the computer system 100 in response to the processor 114 executing one or more sequences of one or more instructions contained in the memory 116. Execution of the sequences of instructions contained in the memory 116 causes the processor 114 to perform methods described herein.

In various embodiments, the computer system 100 may be connected to one or more other computer systems across a network to form a networked system. The network can include a private network or a public network, such as the Internet. The one or more computer systems that store and serve the data may be referred to as servers or the cloud, in a cloud computing scenario. The other computer systems that send and receive data to and from the servers on the cloud may be referred to as client or cloud devices.

The term “computer-readable medium” as used herein refers to any media that participates in providing instructions to the processor 114 for execution. Such a medium may take many forms, including but not limited to, non-volatile media, volatile media, and transmission media. Non-volatile media includes, for example, optical or magnetic disks, such as a storage device 110. Volatile media includes dynamic memory, such as memory 116. Transmission media includes coaxial cables, copper wire, and fiber optics, including the wires that comprise bus 112.

As discussed herein, a test EKG dataset is compared by the processor 114 to a reference EKG dataset. The test EKG dataset is selected from an EKG of a test individual while the reference EKG dataset is selected from an EKG of a reference individual. Both datasets may be stored in the memory 116. The test EKG dataset includes a complete heartbeat that is selected from an EKG captured by the EKG machine 124. The heartbeat may be selected based on being the “best” heartbeat or waveform by the computer system 100 and/or a human operator. The “best” heartbeat is determined based on several different features. As described herein, after capturing a selected heartbeat, a second identical heartbeat is selected and concatenated to the first selected heartbeat to complete the test EKG dataset.

After identifying EKG waveform boundaries and the amplitudes of the selected heartbeat of the test EKG dataset, the processor 114 performs a frequency analysis, such as a Fourier transform on the test and reference EKG datasets. In an embodiment, a plot of a Fourier transform of the reference EKG dataset is already stored in the memory 116. Alternatively, the processor 114 may perform the frequency analysis on the reference EKG dataset as well. In an embodiment, the Fourier transform is a fast Fourier transform (“FFT”). In performing the frequency analysis (e.g., a Fourier transform) on an EKG dataset, the processor 114 converts the EKG dataset from its original time domain to the frequency domain. For example, FIG. 2A illustrates the plotting of a traditional EKG in the time domain where the amplitude of the waveform varies over time, while FIG. 2B illustrates the EKG plot after conversion into the frequency domain, where the amplitude varies by frequency. Note that the second half of the FFT plot is a mirror image of the first half of the FFT plot. Lastly, the processor 114 calculates (or measures) an area under the curve between the frequency domain plot of the EKG dataset of the test individual and the frequency domain plot of the reference EKG dataset (see FIGS. 4 and 5). As described herein, the area under the curve measurement, after it is normalized with respect to a maximum value that equates to a diagnosis of an infarction, provides for a percentage score to be calculated to estimate the extent of ischemia in the test individual. As described herein, a percentage score is used to estimate the extent of ischemia or infarction in the test individual. The higher the area under the curve calculation (and the higher the test EKG dataset diverges from the reference EKG dataset (see FIGS. 4 and 5)), the higher the estimated extent of ischemia or infarction. For example, a percentage score of 100% is an indication that there is an infarction in the test individual, while a percentage score of at least 5-10% is an early indication of ischemia. Thus, percentage scores between 5% and 100% are varying degrees of severity in ischemia and/or infarction.

As illustrated in FIGS. 4 and 5, the processor 114 converts each conventional EKG plot from the time domain to the frequency domain and evaluates the differences between the resulting frequency domain waveforms. FIG. 5 is a magnification of a portion of the plot of FIG. 4. Graphed together, the two EKG waveforms (from the test individual and the reference individual) can be compared. While FIG. 4 illustrates the plotting of the FFT-converted reference EKG and the test EKG, FIG. 5 is an enlarged portion of FIG. 4, illustrating a shorter range of frequencies and with a smaller amplitude scale such that the frequency amplitudes are visible.

While FIG. 5 illustrates frequency amplitudes in the test EKG, FIG. 5 also illustrates that the reference EKG is mostly flat. It is an aspect of the present embodiment that the “area under the curve” between the test individual EKG and the reference EKG defines an estimate of the extent of ischemia. The area under the curve is understood to be a two-dimensional portion of the plot that is defined or bounded by the two EKG waveforms. The area under the curve is also that portion of the test EKG's that has a higher amplitude than the reference EKG. Such measurements or calculations are performed by the processor 114. By measuring the area under the curve, the processor 114 provides an estimate of the extent of ischemia.

With the area under the curve calculated, the processor 114 will “normalize” the calculated results with respect to a maximum possible amplitude (or some other threshold amplitude level), such that a normalized calculated value (e.g., 1) will be at its peak value when the measured area under the curve equals the calculated value equated to an infarction. By calculating the area under the curve and then normalizing it, the processor 114 provides an estimate of the extent of ischemia. In one exemplary embodiment, an area under the curve equal to 1 is an estimate that the test individual has an infarction due to a fully blocked blood vessel. Thus, the calculated value will fall between a minimum value of 0 (no ischemia) and a maximum value of 1 (infarction).

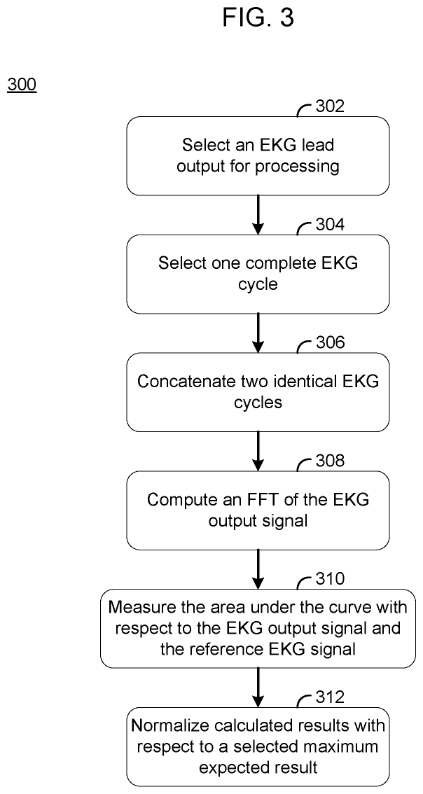

FIG. 3 is an exemplary method 300 for estimating the extent of ischemia in an individual using the computer system 100 illustrated in FIG. 1. As discussed herein, the extent of ischemia can be estimated with a calculated range of 0% to 100% or 0 to 1. The steps to the method 300 of FIG. 3 begin with step 302, where a dataset of electrocardiograms (EKGs) is captured from a test subject via an EKG machine 124. The EKG is used for detecting information from electrical signals that flow through the different muscle tissues of the heart as it is beating. Electrical signals produced by a beating heart are detected between pairs of electrodes. In an embodiment, the EKGs may be captured by a digital EKG device located at a healthcare professional or a doctor's office, a clinic, or a hospital room. In another embodiment, the EKGs may be captured by a wearable device, such as a smart watch worn by an individual.

The EKG data obtained herein may be from an EKG machine having a conventional 12-lead digital EKG device (that includes leads V1, V2, V3, V4, V5, V6, I, II, III, aVF, aVR, and aVL) or a similar measurement device of any number of leads. A measured EKG sequence may represent the voltage measurements as a function of time associated with one of the twelve leads: Lead I, Lead II, Lead III, Lead aVR, Lead aVL, Lead aVF, Lead V1, Lead V2, Lead V3, Lead V4, Lead V5, and Lead V6. Leads V1-V6 may correlate to six different chest positions on an individual. Different individuals may have different or similar sequences associated with each lead. The digital EKG measurement obtained from an individual may include measured voltages obtained from each lead. In an embodiment, the EKG sequence is measured from one of the V1, V2, and V3 leads. In step 304 of FIG. 3, one cycle (i.e., one single heartbeat) is selected and captured (from the EKG plot) for a test EKG dataset. The heartbeat may be selected based on being the “best” heartbeat or waveform by the computer system 100 and/or a human operator. The “best” heartbeat is determined based on several different features. In step 306 of FIG. 3, a second identical cycle (heartbeat) is selected and captured and then concatenated onto the first cycle. In an aspect of the present embodiment, the pair of concatenated identical EKG cycles can be used for additional calculations such that a complete cycle of any phase can be selected within the pair of concatenated EKG heartbeat cycles. Steps 304 and 306 of FIG. 3 may be performed by the processor 114 of the computer system 100. The EKG boundaries for the test individual's EKG cycle computed and/or determined using one or more software programs on the computer system 100, such as MATLAB® or a MATLAB®-based software. The MATLAB® software may also be used to measure the amplitude of the test individual's EKG signal. The measured amplitude is the signal generated by an electrode (of the EKG machine) in response to fluctuations of the electric potential field. As a propagating wave travels closer to an electrode of the EKG machine, the EKG signal amplitude increases, and as it recedes away from the electrode of the EKG machine, the amplitude has reached a maximum negative value and then diminishes in amplitude as the wave-electrode distance increases. Thus, the EKG plot is in the time domain.

In step 308 of FIG. 3, the heartbeat waveform is converted to the frequency domain. That is, the processor 114 calculates the frequency content of the test EKG dataset. As illustrated in FIGS. 2A and 2B, the amplitude/time measurements of the heartbeat are converted to the frequency domain and computed at various harmonic frequencies to the lowest frequency (the fundamental frequency).

In an embodiment, the processor 114 performs the frequency analysis by calculating a Fourier transform on the test EKG dataset. The frequency analysis may be performed using the MATLAB® software via the computer system 100. In an embodiment, the Fourier transform is calculated using a fast Fourier transform (FFT). In other embodiments, other techniques for conducting a frequency analysis or signal processing are performed. The test EKG dataset may be converted into a frequency domain to produce frequency spectra. As illustrated in FIG. 2B, and more specifically in FIGS. 4 and 5, since lower frequency harmonics have higher amplitudes than higher frequency harmonics, the low frequency EKG components are the largest in observed amplitude on the EKG plot. In an embodiment, measurements are taken for a first frequency domain of 0-1000 harmonics (FIG. 4), and for a second frequency domain of 0-600 harmonics (FIG. 5). Note that for FIG. 4, an amplitude scale of 0-10,000 is used, while FIG. 5 uses an amplitude scale of only 0-130. The expanded amplitude scale of FIG. 5 allows for an improved view of the amplitudes of the first few harmonics of the heartbeat waveform. While differences between the reference EKG and the test individual EKG are apparent in the amplitude scale of FIG. 4, they are readily apparent in FIG. 5. As illustrated in FIG. 5, the change in amplitudes in frequency harmonics gets larger in individuals who have ischemia or an infarction. As discussed herein, an analysis of the amplitude differences as compared to the reference provides for an opportunity to estimate the extent of ischemia in the test individual.

In step 310 of FIG. 3, an area under the curve between the test EKG dataset and the reference EKG dataset is calculated or measured. The area under the curve is a measurement of a two-dimensional area between the waveforms of the test EKG dataset and a reference EKG dataset (both in the frequency domain). The area under the curve measurement may be performed using the MATLAB® software via the computer system 100. In an aspect of the embodiment, when the change in amplitudes in frequency harmonics gets larger in individuals, an estimation of the extent of ischemia or infarction is made. In step 312 of FIG. 3, the calculated results are normalized with respect to a selected maximum expected result that equates to a diagnosis of an infarction (such that the calculated value will fall between 0 and 1). The normalized values are thus able to be scaled to a percentage estimate (e.g., 0.0=0% and 1.0=100%). In an embodiment, the largest difference (as determined by an area under the curve measurement) between a test individual's EKG and the reference EKG is classified as a percentage score of 100% when the EKG dataset indicates an infarction (i.e., a percentage score of 100% indicates an infarction is present), while the lowest difference in the area under the curve calculation between a test individual's EKG and the reference EKG is classified as a percentage score of 0% when the EKG dataset indicates no ischemia. The percentage score, indicating the extent of ischemia will vary from a percentage score of 0% (no ischemia) to 100% (an infarction). Thus, a percentage score can be calculated to determine an estimate of the extent of ischemia or infarction for a particular test individual. For example, if test individual receives a score of 0%, then the test individual likely does not have any ischemia, while a score of at least 10% indicates that the test individual likely has a mild case of ischemia. For example, if the score percentage is less than 20%, then the test individual likely does not need to be treated for ischemia. However, if the percentage score is above 10%, but less than 30%, the physician might be able to diagnose ischemia but will be unable to identify where in the body the ischemia is located. However, an attending physician may prescribe a corrective regimen or medication to prevent the ischemia from getting worse. Thus, an estimation of a mild case of ischemia is possible, allowing for early treatment of the ischemia before it can get worse.

The method 300 disclosed herein for estimating the extent of ischemia in a test individual may occur within about two (2) seconds. All the data computed using the method 300 may then be reported to a healthcare professional, such as a physician, for making a final determination regarding the presence and extent of ischemia based on the computed data and estimated score.

As also discussed herein, and illustrated in FIGS. 6 and 7, the differences between an estimated extent of ischemia (i.e., a percentage score of at least 10%) and a diagnosis of hyperkalemia can be difficult to differentiate between because a diagnosis of hyperkalemia also appears to result in a large area under the curve measurement (see FIG. 6). However, as illustrated in FIG. 7, when only a smaller portion of the plot from FIG. 6 is considered by the processor 114, the exemplary enlarged reference EKG plot includes a high amplitude in the first few frequency harmonics (e.g., the first 50 harmonics), while the hyperkalemia EKG plot does not.

It is to be understood that the various embodiments described in this specification and as illustrated in the attached drawings are simply exemplary embodiments illustrating the inventive concepts as defined in the claims. As a result, it is to be understood that the various embodiments described and illustrated may be combined to from the inventive concepts defined in the appended claims.

Thus, exemplary embodiments described herein provide for an estimation of the extent of ischemia in an individual. Because an early warning is not possible when the extent of ischemia in an individual has advanced enough to become an infarction, it is beneficial to estimate an extent of ischemia, such that an “early” diagnosis of ischemia is possible. As described herein, an estimated extent of ischemia in a test individual can be determined by comparing the calculated results of an EKG dataset from the test individual to an EKG dataset from a reference individual. The calculated results include a conversion of the test individual's EKG dataset into the frequency domain. The calculated results (once converted into the frequency domain) also include an area under the curve measurement between the test individual's EKG dataset and the reference EKG dataset. The area under the curve measurement is also normalized with respect to a maximum score that correlates to a diagnosis of an infarction, such that a percentage value range of 0% to 100% may be determined. While a percentage score of 100% correlates to a diagnosis of an infarction, a percentage score between 10% and 20% indicates some extent of ischemia, while a percentage score below 5-10% indicates an estimate of little to no ischemia.

Changes and modifications in the specifically described embodiments can be carried out without departing from the principles of the present invention which is intended to be limited only by the scope of the appended claims, as interpreted according to the principles of patent law including the doctrine of equivalents.

The embodiments of the invention in which an exclusive property or privilege is claimed are defined as follows:

1. A method for estimating the extent of ischemia in a test individual, said method comprising:
using an electrocardiogram (EKG) apparatus, obtaining an EKG of the test individual;
using a computer-based system that is programmed with computer code obtaining a test EKG dataset comprising EKG measurements from the EKG;
using the computer-based system, converting the test EKG dataset to a frequency domain;
using the computer-based system, measuring an area under the curve of the test EKG dataset in the frequency domain with respect to a reference EKG dataset comprising EKG measurements in the frequency domain; and
using the computer-based system, normalizing the area under the curve measurement to calculate a percentage score value to estimate an extent of ischemia in the test individual.

2. The method of claim 1, wherein the reference EKG dataset comprises EKG measurements acquired from an individual that is substantially free of ischemia.

3. The method of claim 1, wherein normalizing the area under the curve measurement is perform with respect to a reference EKG dataset of an individual with a diagnosis of an infarction to define the extent of ischemia in the test individual.

4. The method of claim 3, wherein a diagnosis of infarction is present when the calculated percentage score value is 100%.

5. The method of claim 3, wherein the percentage score value indicates an estimation of ischemia present in the test individual when the calculated percentage score value is at least 10%.

6. The method of claim 1, wherein obtaining a test EKG dataset comprises selecting a first heartbeat waveform from the EKG, and wherein the first heartbeat waveform is a single heartbeat.

7. The method of claim 6, wherein the first single heartbeat waveform is selected by the computer-based system or by an individual.

8. The method of claim 6, wherein obtaining a test EKG dataset comprises selecting a second heartbeat waveform from the EKG that is identical to the first heartbeat waveform and concatenating the second heartbeat waveform to the first heartbeat waveform.

9. The method of claim 1 further comprising calculating a difference between amplitudes of a first 50 harmonics of the test EKG dataset in the frequency domain and a first 50 harmonics of the reference EKG dataset in the frequency domain and estimating an extent of hyperkalemia instead of ischemia when the difference is a reduction in amplitudes of the first 50 harmonics in the test EKG dataset in the frequency domain of at least 40%.

10. A system that is adapted to estimate the extent of ischemia in a test individual, the system comprising:
an electrocardiogram (EKG) apparatus configured to obtain an EKG of the test individual;
a computer-based system programmed with computer code configured to obtain a test EKG dataset comprising EKG measurements from the EKG;
wherein the computer-based system is configured to convert the test EKG dataset to a frequency domain;
wherein the computer-based system is configured to measure an area under the curve of the test EKG dataset in the frequency domain with respect to a reference EKG dataset comprising EKG measurements in the frequency domain; and
wherein the computer-based system is configured to normalize the area under the curve measurement to calculate a percentage score value to estimate an extent of ischemia in the test individual.

11. The system of claim 10, wherein the reference EKG dataset comprises EKG measurements acquired from an individual that is substantially free of ischemia.

12. The system of claim 10, wherein the computer-based system is configured to normalize the area under the curve measurement with respect to a reference EKG dataset of an individual with a diagnosis of an infarction to define the extent of ischemia in the test individual.

13. The system of claim 12, wherein a diagnosis of infarction is present when the calculated percentage score value is 100%.

14. The system of claim 12, wherein the percentage score value indicates an estimation of ischemia present in the test individual when the calculated percentage score value is at least 10%.

15. The system of claim 10, wherein the computer-based system is configured to select a first heartbeat waveform from the EKG for the test EKG dataset, and wherein the first heartbeat waveform is a single heartbeat.

16. The system of claim 15, wherein the computer-based system is configured to select a second heartbeat waveform from the EKG for the test EKG dataset, wherein the second heartbeat waveform is identical to the first heartbeat waveform, and wherein the computer-based system is configured to concatenate the second heartbeat waveform to the first heartbeat waveform.

17. The system of claim 10, wherein the computer-based system is configured to calculate a difference between amplitudes of a first 50 harmonics of the test EKG dataset in the frequency domain and a first 50 harmonics of the reference EKG dataset in the frequency domain and to estimate an extent of hyperkalemia instead of ischemia when the difference is a reduction in amplitudes of the first 50 harmonics in the test EKG dataset in the frequency domain of at least 40%.
